(19) United States
(12) Patent Application Publication
(10) Pub. No.: US 2024/0207317 A1
(43) Pub. Date: Jun. 27, 2024

(54) METHOD

(71) Applicant: Babraham Institute, Cambridge (GB)

(72) Inventors: Martin TURNER, Cambridge (GB); Twm Joseff MITCHELL, Cambridge (GB); Georg PETKAU, Cambridge (GB); David J. TURNER, Cambridge (GB)

(21) Appl. No.: 18/539,581

(22) Filed: Dec. 14, 2023

Related U.S. Application Data
(60) Provisional application No. 63/387,379, filed on Dec. 14, 2022.

(51) Int. Cl. A61K 35/17; A61K 39/00; A61P 31/16; C12N 5/0783; C12N 15/113

(52) U.S. Cl. CPC A61K 35/17; A61K 39/4611; A61K 39/4631; A61K 39/464838; A61P 31/16; C12N 5/0638; C12N 15/1136

(57) ABSTRACT

The invention relates to a method of enhancing cytotoxic T lymphocyte (CTL) activation and/or proliferation comprising inhibiting or reducing the activity of one or more post-transcriptional regulators of gene expression. Examples of such post-transcriptional regulators include RNA-binding proteins. Also provided is a CTL with enhanced activity and/or proliferative capacity obtainable by the methods defined herein, and a method of treating a disease or disorder in a subject in need thereof comprising administering said CTL with enhanced activity and/or proliferative capacity.

### FIELD OF THE INVENTION

The invention relates to a method of enhancing cytotoxic T lymphocyte (CTL) activation and/or proliferation comprising inhibiting or reducing the activity of one or more post-transcriptional regulators of gene expression. Examples of such post-transcriptional regulators include RNA-binding proteins. Also provided is a CTL with enhanced activity and/or proliferative capacity obtainable by the methods defined herein, and a method of treating a disease or disorder in a subject in need thereof comprising administering said CTL with enhanced activity and/or proliferative capacity.

### BACKGROUND OF THE INVENTION

CD8+ T cells are instrumental for the clearance of pathogen infected or malignant cells and the provision of immune memory. Upon T cell receptor (TCR) sensing of peptide presented by MHC-I, naïve CD8+ T cells exit quiescence and engage a differentiation program to form cytotoxic T-lymphocytes (CTLs) accompanied by massive clonal expansion. High affinity antigen overrides inhibitory mechanisms which ensure the quiescent state of the CD8+ T cell and promotes growth and cell cycle entry (Bennett, Udupa & Turner (2020) Int. J. Mol. Sci., 21(24):9773, doi: https://doi.org/10.3390/ijms21249773; and Gett et al. (2003) Nat. Immunol., 4(4):355-360, DOI: https://doi.org/10.1038/ni908). Co-stimulatory signals, termed “signal 2”, from CD28 are critical for lowering the activation threshold-especially when TCR stimulation is suboptimal. The amount and duration of TCR stimulation correlates with the clonal expansion of antigen-specific cells (Obst (2015) Front. Immunol., 6:563, DOI: https://doi.org/10.3389/fimmu.2015.00563). However, weak TCR signals are sufficient to induce a full differentiation program, albeit more slowly (Zehn, Lee & Bevan (2009) Nature, 458(7235):211-214, doi: https://doi.org/10.1038/nature07657; and Richard et al. (2018) Nat. Immunol., 19(8):849-858, doi: https://doi.org/10.1038/s41590-018-0160-9). The cytotoxic effector differentiation program is installed early after activation with estimates ranging between 2- and 48-h after antigen stimulation (Obst (2015)). Persistent exposure to IL-2, IL-12 and type-I interferon further shapes the response of CD8+ T cells by providing critical survival factors and promoting effector differentiation (Pipkin et al. (2010) Immunity, 32(1):79-90, doi: https://doi.org/10.1016/j.immuni.2009.11.012; Starbeck-Miller, Xue & Harty (2014) J. Exp. Med., 211(11):105-120, doi: https://doi.org/10.1084/jem.20130901; and Joshi et al. (2007) Immunity, 27(2):281-295, doi: https://doi.org/10.1016/j.immuni.2007.07.010). The field of adoptive cell therapy for cancer, with the development of chimeric antigen receptor (CAR) programmed T cells as an antigen specific targeted tumour therapy is a new era in medicine. However, these cell-based medicines are still ineffectual in many cancers. Therefore, there is a great need to enhance the effector functions of these cells without toxic side effects.

### SUMMARY OF THE INVENTION

According to a first aspect of the invention, there is provided a method of enhancing cytotoxic T lymphocyte (CTL) activation and/or proliferation, said method comprising inhibiting or reducing the activity of one or more post-transcriptional regulators of gene expression.

In one embodiment, the method enhances CTL activation independently of enhancing proliferation. In a further embodiment, the one or more post-transcriptional regulators of gene expression are one or more RNA-binding proteins.

According to a further aspect of the invention, there is provided a cytotoxic T lymphocyte (CTL) with enhanced activity and/or proliferative capacity obtainable by the method defined herein.

In a yet further aspect, there is provided a method of treating a disease or disorder in a subject in need thereof, said method comprising administering the CTL with enhanced activity and/or proliferative capacity as defined herein to the subject, wherein said CTL recognises and is reactive to an antigen associated with the disease or disorder.

### BRIEF DESCRIPTION OF THE FIGURES

FIG. 1: A) CTV dilution by WT (open histograms) and dKO (filled histograms) T cells stimulated for 72 hours, with indicated concentrations of anti CD3 plate bound antibody. B) Total numbers of cells recovered per generation after 72h of stimulation with anti CD3 antibodies. Data from n=3 biological replicates is presented as mean values +/−SD. C) CTV dilution by WT and dKO T cells after 72 hours in the presence of 5 μg/ml plate bound anti CD3 (left panel) and with the addition of 10 μg/ml plate bound anti CD28 (right panel). D) Cell numbers per generation after 72 hours with plate bound anti CD3 and varying amounts of plate bound anti-CD28 from WT and dKO cells. Data from n=3 biological replicates is presented as mean values+/−SD.

FIG. 2: A) In vitro killing of N4 peptide and B) V4 peptide loaded EL4 cells after three hours at indicated CTL to EL4 ratios, using CTLs with CD4^{cre} mediated deletion of ZFP36 and ZFP36L1 and WT CTLs. Data is represented as mean values+/−SD. Statistical significance was determined by one-way ANOVA followed by Tukey's test for multiple comparisons.

FIG. 3: A) FACS plots are pre-gated on live day 9 memory-like CD8+ T cells stimulated for 3 hours with 10-10 M of SIINFEKL (N4) peptide (n=3). B) Estimated protein copy number per cell in memory-like CD8+ T cells stimulated for 8 hours with 10-10 M N4 peptide (n=4).

FIG. 4: Intersectionality of two genetic screens in mouse CD8 T cells identifies regulators of T cell activation.

FIG. 5: A) Western blot of CTL following Cas9-RNP meditated mutation of Cnot9 and Ddx6 in naïve OT-I cells prior to in vitro CTL generation. B) Peptide-specific killing by CTL; data from each independent experiment represent the mean±standard error of 2 or 3 technical replicates.

FIG. 6: A) Volcano plot showing-Log 2 fold change of protein abundance between Ctrl and DDX6 KO CTL prior to stimulation. A range of cytotoxic proteins are highlighted. Figures on volcano plot in bold show number of distinct proteins which are significantly increased or decreased in abundance (Threshold: Log 2FC >0.588 or <-0.588, with accompanying P value <0.05). B) Heat map showing Log 2FC of cytotoxic protein copy numbers detected in Ctrl and DDX6 KO CTL prior to stimulation (0h) or following one- or three-hour stimulation with N4 peptide. P values were calculated using a paired Student's t test, N=4. C-D) Graphs show the geometric mean fluorescence intensity (MFI) of the Pore-forming protein Perforin and cytotoxic serine protease Granzyme-B detected in Ctrl and DDX6 KO CTL by flow cytometry. P values were calculated using a paired Student's t test, N=7. E) Kinetic graph shows bodyweight loss relative to time of infection of recipient mice on Days 4-10 post-infection. Error bars show standard error from the mean, and P value was calculated using Two-Way ANOVA with Šídak's multiple comparisons test, N=6. F) Graphs show the total number of CD45.1⁺OT-I cells recovered from the Spleens and Lungs of recipient mice Day 14 post-infection. P values were calculated using a paired Student's t test, N=4.

### DETAILED DESCRIPTION OF THE INVENTION

The present invention is based on the discovery of a new pathway that limits the cytotoxicity of T cells and their ability to kill target cells. In particular, it has been previously reported that the RNA-binding proteins (also referred to as RBPs) ZFP36 and ZFP36L1 act to limit the differentiation of naïve CD8+ T cells to cytotoxic T lymphocytes (CTLs) and the subsequent activation of and cytokine release by CD8+CTLs (Petkau et al. (2022) Nat. Commun., 13(2274), doi: https://doi.org/10.1038/s41467-022-29979-x). The inventors have herein identified, using a CRISPR screen, additional post-transcriptional regulators of gene expression (including several RBPs) that limit T cell function and activity. These findings have significant potential in adoptive cell therapies, for example for the treatment of cancer. In particular and as provided by the present invention, the ability to augment the effector functions of engineered T cells such as those with chimeric antigen receptors (CARs) which recognise and react to specific tumour antigens, without toxic side effects, has great utility in these cell therapies which are often ineffectual in many cancers.

Thus, according to a first aspect of the invention, there is provided a method of enhancing cytotoxic T lymphocyte (CTL) activation and/or proliferation, said method comprising inhibiting or reducing the activity of one or more post-transcriptional regulators of gene expression.

References herein to “enhancing CTL activation and/or proliferation” refer to an increase or promotion of the cytotoxic activity and/or effector functions of said CTL, and include wherein the differentiation of naïve T cells to CTLs is enhanced/increased. In one embodiment, enhanced activity may be increased expression of proinflammatory cytokines by the CTL, such as TNFα, IFNγ and/or IL-2, and/or other effector cytokines, such as granzyme B and/or perforin. Thus, in a particular embodiment, the effector functions of the CTL are enhanced. In another particular embodiment, the release of granzyme B, perforin, TNFα and/or IFNγ by the CTL are enhanced. Measurement of proinflammatory/effector cytokines may be, for example, by ELISA, including multiplex ELISA, which measures the amount of cytokine released/secreted by cells or by intracellular flow cytometry following treatment of the cells with a compound which prevents their secretion (e.g. brefeldin A which traps cytokines in the Golgi apparatus during their synthesis). Alternatively, the measurement of proinflammatory/effector cytokine expression may be by measuring the abundance of mRNA transcripts encoding the proinflammatory/effector cytokines, such as by quantitative PCR (qPCR) or sequencing. However, as will be appreciated by the disclosures herein, due to the function of post-transcriptional gene expression regulators (e.g. RNA-binding proteins) in affecting the stability of mRNA transcripts or affecting the translation efficiency of mRNA transcripts without altering their stability, measurement of the proinflammatory/effector cytokine protein levels is preferred. In a further embodiment, enhanced CTL activity may also or alternatively be an enhanced ability of the CTL to kill a target cell, such as a cell loaded with a peptide which the CTL recognises and is reactive to (e.g. a tumour or virus-infected cell). Thus in a particular embodiment, the killing functions of the CTL are enhanced. For example, when the CTL is an engineered T cell, such as a chimeric antigen receptor (CAR) T cell, enhanced activity may be measured by enhanced killing of a target cell expressing or loaded with the specific antigen which the engineered T cell receptor (e.g. CAR) recognises. In one embodiment, the target cell is a cancer or tumour cell. In a yet further embodiment, enhanced CTL activity may also or alternatively be enhanced differentiation of naïve T cells to CTLs. Such differentiation may be measured, for example, by increased expression and/or activity of IL-2, the canonical NF-κB pathway, the non-canonical NF-κB pathway (e.g. NF-κB2), NOTCH1, Eomesodermin (EOMES), granzyme B, IRF4 and/or IRF8. In a still further embodiment, enhanced CTL activity may also or alternatively be reduced dependence on co-stimulation, in particular CD28 co-stimulation. For example, a lower amount and/or shorter time period of CD28 co-stimulation is required to fully activate the CTL with enhanced activity compared to a CTL not obtained by the methods described herein. In a yet further embodiment, enhancement of CTL activity may comprise reduced exhaustion of the CTL. The exhaustion phenotype of a CTL can be measured by the presence of inhibitory receptors on the cell surface (e.g. PD-1, TIM-3 and LAG-3) and by a reduction in the cell's ability to perform effector functions (e.g. it has reduced killing potential and effector cytokine production). In another embodiment, the proliferation of CTLs is enhanced. Enhanced proliferation can be measured by known techniques in the art, including, without limitation, cell counting after a defined period of time, dilution of a cell surface protein stain (e.g. carboxyfluorescein succinimidyl ester (CFSE) or CellTrace™ dyes), increased DNA content or expression of a proliferation marker (e.g. Ki67 and/or cell division checkpoint proteins). As will be readily appreciated, any enhancement, increase or promotion of CTL activity and/or proliferation described herein is relative to a lymphocyte/CTL in which the activity of one or more post-transcriptional regulators of gene expression is not inhibited or reduced.

In certain embodiments, the method enhances CTL activation independently of enhancing proliferation. Thus in a further embodiment, the method enhances CTL activation without enhancing proliferation.

### Cytotoxic T Lymphocyte Functions

Cytotoxic T lymphocytes (CTLs) are T cells that kill cancer cells, cells that are infected by intracellular pathogens (such as viruses or intracellular bacteria) or cells that may be damaged in other ways. In other words, CTLs recognise and react to cells harbouring foreign, abnormal or altered intracellular protein products. T cells express T cell receptors (TCRs) that recognise antigen presented as short peptides in the context of MHC on the surface of a target cell. Antigens produced intracellularly are bound and presented by MHC class I and are then recognised by CD8+ T cells. Thus in one embodiment, the cytotoxic T lymphocyte (CTL) is a CD8+ T cell. CD8 is a glycoprotein which binds to the constant portion of MHC class I and is required for TCR binding to the MHC-peptide complex on target cells. Full activation of CTLs requires co-stimulation, such as by binding with CD28 on the CTL, together with TCR binding. CD28 binds to CD80 or CD86 on target cells, although the requirement for co-stimulation can be reduced by cytokines from T helper cells (often CD4+ T cells). In another embodiment, the cytotoxic T lymphocyte (CTL) is a CD4+ T cell. The present inventors have previously reported the surprising finding that by inhibiting/reducing the activity of certain RNA-binding proteins in CTLs, the requirement for co-stimulation is reduced (Petkau et al. (2022)). Thus in a further embodiment, the CTL with enhanced activity and/or proliferative capacity, such as the CTL produced according to the methods described herein, has a reduced requirement for co-stimulation, in particular by CD28, compared to a CTL in which the activity of one or more post-transcriptional regulators of gene expression is not inhibited or reduced.

Post-Transcriptional Gene Expression Regulators (e.g. RNA-Binding Proteins)

Post-transcriptional regulation of gene expression is the control of expression at the RNA level (e.g. messenger RNA (mRNA)) between the transcription phase and the translation phase of gene expression. After transcription, the stability, cellular location and translation of mRNA transcripts is regulated by means of RNA-binding proteins (RBPs) that control events such as alternative splicing, nuclear degradation, processing, nuclear export, sequestration in P- or S-bodies (S-bodies may also be known as stress granules) for storage or degradation, and eventually the translation of the mRNA. These proteins comprise an RNA recognition motif that binds a specific sequence or secondary structure of the transcripts, typically at the 5′ and/or 3′ untranslated regions (UTRs) of the transcript. Thus in a particular embodiment, the one or more post-transcriptional regulators of gene expression are one or more RNA-binding proteins. In a further embodiment, the one or more RNA-binding proteins regulate the stability and/or translation efficiency of an mRNA transcript. In a yet further particular embodiment, the one or more RNA-binding proteins reduce or inhibit the stability and/or translation efficiency of the mRNA transcript.

The role for certain RNA-binding proteins in T cell responses is known. For example, the ability of Roquin (Rc3h1) and Regnase1 (Rc3h12a) to limit CD8+ T cell responses has been linked to the repression of TCR signalling and cytokine production (Chang et al. (2012) Cells J. I., 189(2):701-710, doi: https://doi.org/10.4049/jimmunol.1102432; and Fu & Blackshear (2017) Nat. Rev. Immunol., 17(2):130-143, doi: https://doi.org/10.1038/nri.2016.129), and their absence leads to T cell hyperactivation and autoimmune/inflammatory disease. The ZFP36 family of RNA-binding proteins bind AU-rich elements (AREs) present in the 3′ untranslated region (3′UTR) of mRNAs and can effect different outcomes promoting RNA decay (Fu & Blackshear (2017)), suppressing translation (Salerno, Turner & Wolkers (2020) Trends Immunol., 41(3):240-254 (2020), doi: https://doi.org/10.1016/j.it.2020.01.001; Moore et al. (2018) eLife, 7:e33057, doi: https://doi.org/10.7554/eLife.33057; and Bell et al. (2006) Dev. Dyn., 235(11):3144-3155, doi: https://doi.org/10.1002/dvdy.20949) or directing localised translation (Ma & Mayr (2018) Cell, 175(6):1492-1506.e19, doi: https://doi.org/10.1016/j.cell.2018.10.007) which are cell-context-specific (Sneezum et al. (2020) Front. Immunol., 11:1398, doi: https://doi.org/10.3389/fimmu.2020.01398). Of the three ZFP36 gene-family members expressed by CD8+ T cells, ZFP36L2 is present in naïve and memory cells, while ZFP36 and ZFP36L1 are induced rapidly and transiently following TCR stimulation Moore et al. (2018)). Zfp36-deficient mice show heightened immune responses and develop a severe autoimmune syndrome attributable to its function in myeloid cells (Fu & Blackshear (2017); and Moore et al. (2018)). An enhanced CD8 response in Zfp36-deficient mice has been linked to the excessive production by myeloid cells of IL-27, of which the p28 subunit is a direct target of ZFP36 (Wang et al. (2017) Nat. Commun., 8:867, doi: https://doi.org/10.1038/s41467-017-00892-y). In quiescent memory CD8+ T cells the ZFP36-paralog ZFP36L2 suppresses the translation of cytokine mRNA (Salerno et al. (2018) Nat. Immunol., 19(8):828-837, doi: https://doi.org/10.1038/s41590-018-0155-6). However, to date no studies have utilised screening to identify additional RNA-binding proteins that regulate CTL cytokine expression and activation.

Thus in one embodiment, the RNA-binding protein is a member of the ZPF36 family, in particular ZFP36 and/or ZPF36L1 (also known as tristetraprolin/TTP/TIS11 and TIS11B, respectively). As demonstrated by the data in Petkau et al. (2022; the contents of which are hereby incorporated in their entirety) and results presented herein, ZPF36 and ZPF36L1 limit the rate of differentiation of activated naïve CD8+ T cells and the potency of the resulting CTLs. Thus, the inhibition or reduction of ZFP36 and ZFP36L1 activity in CD8+ T cells enhances/increases the activation and/or proliferation of CTLs, enhancing their cytotoxic effector functions, in particular enhancing their ability to kill target cells, enhancing effector cytokine production and reducing the requirement for CD28 co-stimulation. In a further embodiment, the one or more post-transcriptional regulators of gene expression, such as the RNA-binding protein, is selected from any one or more of those listed in Table 1. The RNA-binding protein genes listed in Table 1 have been identified in CD8+ T cells using a CRISPR knock out screen, and as demonstrated by the results herein their inhibition/reduction of their activity led to increased production of the effector cytokines TNFα and IFNγ, and/or the levels of cytotoxic proteins in stimulated CTLs. Thus, the inhibition or reduction of the activity of the RNA-binding proteins listed in Table 1 enhances/increases the activation of CTLs, enhancing the effector functions.

TABLE 1
RNA-Binding Protein Genes Enriched in a Genetic Screen of CD8
T cell Activation, where Gene Knock Out Promoted Activation

| RNA-binding | Overall RANK After | |
|---|---|---|
| Protein | Removal of Controls | Z-Score |
| Ago2 | 4 | −9.712 |
| Naca | 5 | −8.580 |
| Cnot9 | 7 | −7.131 |
| Tnrc6a | 8 | −7.096 |
| Cnbp | 9 | −6.780 |
| Btf3 | 10 | −6.655 |
| Sugp1 | 11 | −6.636 |
| Ddx6 | 12 | −5.914 |
| Denr | 13 | −5.392 |
| Ptbp1 | 14 | −5.326 |
| Xpo6 | 15 | −5.323 |
| Eif4b | 17 | −5.117 |
| Sumo2 | 18 | −4.956 |
| Xpo5 | 19 | −4.699 |
| Hnrnpa2b1 | 20 | −4.681 |
| Hnrnpf | 21 | −4.613 |
| Ybx1 | 22 | −4.443 |
| Dicer1 | 23 | −4.329 |
| Dhx30 | 24 | −4.325 |
| Scaf8 | 25 | −4.221 |
| Zfp36l2 | 26 | −4.162 |
| Zranb2 | 27 | −4.129 |
| Cnot6l | 28 | −4.100 |
| Trip12 | 29 | −4.062 |
| Hexim1 | 30 | −3.841 |
| Zc3h4 | 31 | −3.829 |
| Larp4 | 32 | −3.755 |
| Cnot4 | 34 | −3.679 |
| Rbm38 | 35 | −3.647 |
| Atp5a1 | 36 | −3.631 |
| Cpsf6 | 37 | −3.575 |
| Hnrnpm | 38 | −3.564 |
| Nmt1 | 39 | −3.551 |
| Rbm12 | 40 | −3.473 |
| Nufip2 | 41 | −3.471 |
| Ddx28 | 43 | −3.438 |
| Rbm26 | 44 | −3.388 |
| Zc3hav1 | 45 | −3.364 |
| Ascc3 | 46 | −3.248 |
| Mbnl1 | 47 | −3.210 |
| Srsf10 | 48 | −3.126 |
| Srek1 | 49 | −3.085 |
| Atp5b | 50 | −3.051 |
| Rbm5 | 51 | −3.030 |

The RNA-binding protein genes identified in Table 1 as enriched for inhibiting CD8+ T cell activation (i.e. wherein knock out promoted activation) were measured by increased expression/release of effector cytokines TNFα and IFNγ.

In certain embodiments, the one or more post-transcriptional regulators of gene expression is one or more components of the CCR4-NOT complex. The CCR4-NOT complex is the principal eukaryotic deadenylase and sets a non-specific background level of deadenylation for cellular mRNAs (Raisch et al. (2019) Nat. Commun., 10(3173), doi: https://doi.org/10.1038/s41467-019-10 11094-z; and Collart (2016) Wiley Interdiscip. Rev.: RNA, 7(4):438-454, doi: https://doi.org/10.1002/wrna.1332). Trans-acting factors recruit the CCR4-NOT complex to their target transcripts, thus promoting targeted deadenylation and facilitating subsequent RNA decay (Du et al. (2016) Nat. Commun., 7(12626), doi: https://doi.org/10.1038/ncomms12626). The CCR4-NOT complex can be employed as an adaptor and in turn recruit RNA-binding proteins involved in the repression of translation initiation. For example, miRNAs in ribonucleic protein (RNP) complexes recruit the CCR4-NOT complex to their targets, leading to translational repression as well as deadenylation followed by decay (Gebert & MacRae (2019) Nat. Rev. Mol. Cell Biol., 20:21-37, doi: https://doi.org/10.1038/s41580-018-0045-7). As shown hereinbefore, several members of the CCR4-NOT complex were identified in Table 1 as being involved in the limiting of CTL activation, in particular CD8+CTL activation and effector functions. In a particular embodiment, the one or more post-transcriptional regulators of gene expression are selected from one or more of: DDX6; HNRNPF; CNOT4; CNOT6L; and/or CNOT9. In one embodiment, the one or more component of the CCR4-NOT complex is DDX6. The Ddx6 gene (DEAD-box helicase 6) encodes an RNA helicase, a motor protein that moves directionally along a nucleic acid phosphodiester backbone, separating two hybridized strands, using energy from ATP hydrolysis. As shown herein, knock out of Ddx6 enhanced the cytotoxic functions of CTL through increased levels of cytotoxic proteins in these cells and results in protection from influenza virus infection in vivo. Thus, DDX6 limits cytotoxic protein expression in CTL and response to infection. In a further embodiment, the one or more component of the CCR4-NOT complex is hnRNPF. The Hnrnpf gene encodes heterogeneous nuclear ribonucleoprotein F, which is involved in mRNA metabolism, transport and processing, and has recently been associated with autoimmune disease Kakiuchi et al. (2020) Nature, 577(7789):260-265, doi: https://doi.org/10.1038/s41586-019-1856-1). In yet further embodiments, the one or more component of the CCR4-NOT complex is one or more of CNOT4; CNOT6L; and/or CNOT9. The Cnot-4 and Cnot-6/genes encode functional components of the CCR4-NOT complex with ubiquitin ligase activity (CNOT4) and a paralog of CNOT6 (CNOT6L) which has 3′-5′ exonuclease activity and has been reported to prefer polyadenylated substates (i.e. mRNAs), respectively, while the Cnot-9 gene encodes a core component of the CCR4-NOT complex.

In some embodiments, the activity of the one or more post-transcriptional regulators of gene expression (e.g. RNA-binding proteins) are inhibited and/or reduced. Such inhibition/reduction of activity may be at the protein level and comprise inhibition of catalytic activity (e.g. the inhibition of ATP hydrolysis and thus helicase activity in the case of DDX6) and/or inhibition or reduction of protein binding. In the context of the present invention, such protein binding may comprise binding of the RNA-binding protein to target mRNA transcripts and/or binding to other proteins within a complex, such as the CCR4-NOT complex. In one embodiment, inhibition or reduction in activity is inhibition/reduction of binding to a target mRNA transcript. In another embodiment, inhibition or reduction is inhibition/reduction of binding to proteins within a complex, such as the CCR4-NOT complex. Inhibition/reduction of activity may alternatively be at the gene level or gene expression level, e.g. by deleting or disrupting the gene encoding the one or more post-transcriptional gene expression regulator, or by preventing the translation of an mRNA transcript encoding said one or more post-transcriptional gene expression regulator. Thus in one embodiment, inhibition or reduction in activity is deletion or disruption of the gene encoding the one or more one or more post-transcriptional gene expression regulator. In a particular embodiment, disruption of said gene comprises deletion, knock out or introduction of an indel. Methods of disruption, deletion and knock out of a gene are well known in the art and include, without limitation, recombinase-driven deletion of a gene. Disruption, knock out and/or introduction of an indel may also be performed using, for example, CRISPR-Cas which results in the repair of a double-stranded break made by the targeted Cas9 (or other Cas protein, such as Cas12a) by means of non-homologous end joining (NHEJ). NHEJ often results in random deletions or insertions at the repair site, which may disrupt or alter gene functionality, thus giving the ability to generate targeted random gene disruption.

Thus, any method of making specific, targeted double strand breaks in the gene encoding the one or more one or more post-transcriptional gene expression regulator may be used in the method of the invention. Any one or more of ZFNs, TALENs and/or CRISPR-Cas systems or any derivative thereof may be used. In a particular embodiment, a CRISPR-Cas system is used together with one or more single-guide RNA (sgRNA) which targets the Cas endonuclease to one or more gene encoding the post-transcriptional gene expression regulator. Thus in certain embodiments, the one or more sgRNA comprises a sequence which is complementary to one or more RNA-binding protein genes listed in Table 1. In a further embodiment, the one or more sgRNA comprises a sequence which is complementary to one or more components of the CCR4-NOT complex, such as one or more of: DDX6; HNRNPF; CNOT4; CNOT6L; and/or CNOT9.

The machinery for disrupting the gene encoding the one or more one or more post-transcriptional gene expression regulator may be supplied on one or more vectors. A “vector” is a nucleic acid molecule, such as a DNA molecule, which is used as a vehicle to artificially transfer exogenous genetic material into a cell. The vector is generally a nucleic acid sequence that consists of an insert and a larger sequence that serves as the “backbone” of the vector. The vector may be in any suitable format, including plasmids, minicircle, linear DNA or a single-stranded AAV template. The vector comprises at least the gene for the machinery required for generating a double-stranded break. Optionally, the vectors also possess an origin of replication (ori) which permits amplification of the vector, for example in bacteria. Additionally or alternatively, the vector includes selectable markers such as antibiotic resistance genes, genes for coloured markers and/or suicide genes.

Vectors include but are not limited to plasmids, cosmids, viruses (bacteriophage, animal viruses and plant viruses) and artificial chromosomes (e.g. yeast artificial chromosomes or YACs).

In one embodiment, the vector(s) is a viral vector. The viral gene delivery system may be an RNA-based or DNA-based viral vector. Viral vectors include retroviral vectors (e.g., lentiviral vectors, such as those derived from HIV-1, HIV-2, SIV, BIV, FIV etc.), gammaretroviral vectors, adenoviral (Ad) vectors (including replication competent, replication deficient and gutless forms thereof), adeno-associated virus-derived (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumour virus vectors, Rous sarcoma virus vectors and Sendai virus vectors. In a further embodiment, the viral vector(s) is selected from: a lentiviral vector, an adeno-associated virus vector or a Sendai virus vector. In a yet further embodiment, the viral vector(s) is a lentiviral vector.

Lentiviral vectors are well known in the art. Lentiviral vectors are complex retroviruses capable of integrating randomly into the host cell genome, which, in addition to the common retroviral genes gag, pol, and env, contain other genes with regulatory or structural function (e.g. accessory genes Vif, Nef, Vpu, Vpr). Lentiviral vectors have the advantage of being able to infect non-dividing cells and can be used for both in vivo and ex vivo gene transfer and expression of nucleic acid sequences. For example, a recombinant lentiviral vector is capable of infecting a non-dividing cell wherein a suitable host cell is transfected with two or more vectors carrying the packaging functions, namely gag, pol and env, as well as rev and tat.

In another embodiment, the vector(s) is a plasmid. The plasmid(s) may be episomal. Episomal vectors are able to introduce large fragments of DNA into a cell but are maintained extra-chromosomally, replicated once per cell cycle, partitioned to daughter cells efficiently, and elicit substantially no immune response. Alternatively, an Epstein-Barr virus (EBV)-based episomal vector, a yeast-based vector, an adenovirus-based vector, a simian virus 40 (SV40)-based episomal vector, or a bovine papilloma virus (BPV)-based vector may be used.

In certain embodiments, the gene encoding the one or more post-transcriptional regulators of gene expression is endogenous to the CTL. In other words, the gene encoding the post-transcriptional gene expression regulator is endogenous to the CTL with enhanced activation and/or proliferation defined herein. Thus in some embodiments, the post-transcriptional gene expression regulator effects the post-transcriptional expression of mRNA transcripts in the CTL with enhanced activity and/or proliferation, e.g. the mRNA transcripts of effector cytokines. According to these embodiments, the inhibition/reduction of the activity of the one or more post-transcriptional gene expression regulator is cell-intrinsic (e.g. autonomous) to the CTL with enhanced activity and/or proliferation. In alternative embodiments, the gene encoding the one or more post-transcriptional regulators of gene expression is exogenous to the CTL. For example, the gene encoding the post-transcriptional gene expression regulator is endogenous to a different cell (e.g. a helper cell) to the CTL with enhanced activation and/or proliferation. Thus in one embodiment, the post-transcriptional gene expression regulator effects the post-transcriptional expression of mRNA transcripts in a helper cell, e.g. mRNA transcripts of cytokines such as IL-2, IL-12 and type-I interferon. Said cytokines bring about enhanced or increased activity and/or proliferation in the CTL. According to these alternative embodiments, inhibition/reduction of the activity of the one or more post-transcriptional gene expression regulator is cell-extrinsic (e.g. non-autonomous) to the CTL with enhanced activity and/or proliferation.

### Engineered T Cells

In certain embodiments, the CTL is an engineered T cell. In a particular embodiment, the CTL is a chimeric antigen receptor (CAR) T cell. CAR T cells are known in the art and are engineered to express a T cell receptor (TCR) which recognises and is reactive to a particular antigen. They can be generated from either or both CD4+ and CD8+ T cells. They find particular utility in anti-cancer therapy and combine antibody-based specificity for an antigen with a T cell receptor-activating intracellular domain. Thus, the term “CAR” as used herein refers to an engineered receptor comprising an extracellular domain (usually in the form of or derived from a single chain antigen-binding fragment of an antibody (scFv)), optionally a spacer region, a transmembrane region and one or more intracellular effector domains. CARs are genetically introduced into T cells to redirect their specificity for a particular cell-surface antigen, resulting in a CAR T therapeutic. The “transmembrane domain” spans the cell membrane of the T cell, and the “intracellular effector domain” (also referred to as the “signalling domain”) is responsible for intracellular signalling following the binding of the antigen binding domain to the antigen/target.

In certain embodiments, the engineered T cell (e.g. CAR T cell) recognises and is reactive to a tumour antigen. In other embodiments, the engineered T cell (e.g. CAR T cell) recognises and is reactive to a cancer antigen.

Thus in a further aspect of the invention, there is provided a cytotoxic T lymphocyte (CTL) with enhanced activity and/or proliferative capacity obtainable by the method defined herein. In a certain embodiment, the enhanced activity is enhanced cytotoxic activity, such as by increased levels of cytotoxic proteins in the CTL (e.g. perforin and/or granzyme-B). Thus, the CTL with enhanced activity may comprise increased levels of cytotoxic proteins, such as perforin and/or granzyme-B. In a particular aspect, there is provided an engineered CTL (e.g. comprising a CAR) with enhanced activity and/or proliferative capacity obtainable by the method defined herein. In one embodiment, the engineered CTL with enhanced activity and/or proliferative capacity is a CD8+CAR T cell. In another embodiment, the engineered CTL with enhanced activity and/or proliferative capacity is a CD4+CAR T cell. In a further embodiment, the engineered CTL (which may comprise a CAR) has enhanced cytotoxic activity. In a yet further embodiment, the engineered CTL may comprise increased levels of cytotoxic proteins, such as perforin and/or granzyme-B.

### In Vitro and Therapeutic Methods

In certain embodiments, the method of enhancing cytotoxic T lymphocyte (CTL) activation and/or proliferation defined herein is performed in vitro. In a further embodiment, the method is performed in an in vitro cell culture. In one embodiment, the method is performed in vitro and the CTL with enhanced activity and/or proliferation obtainable by or produced by the method is used in a method of treatment, such as for cancer.

Thus in a further aspect of the invention, there is provided a cytotoxic T lymphocyte (CTL) with enhanced activity and/or proliferative capacity as described hereinbefore obtainable by the method defined herein. In a particular aspect, there is provided a CD8+CTL with enhanced activity and/or proliferative capacity as described hereinbefore obtainable by the method defined herein. In some embodiments, the CTL is comprised in a pharmaceutical composition, optionally further comprising one or more pharmaceutically acceptable carriers, diluents and/or excipients. Thus in a yet further aspect of the invention, there is provided a pharmaceutical composition comprising the CTL with enhanced activity and/or proliferative capacity as described hereinbefore obtainable by the method defined herein.

In one embodiment, the CTL with enhanced activity and/or proliferative capacity as defined herein is produced by the method in vitro, such as in a cell culture. In an alternative embodiment, the CTL is produced by the method in vivo. According to this alternative embodiment, the method of enhancing CTL activation and/or proliferation defined herein comprises the steps of: (i) providing a CTL, such as an engineered (e.g. CAR) T cell, in vivo in a subject; and (ii) inhibiting or reducing the activity of one or more post-transcriptional regulators of gene expression in said CTL in vivo. Thus in some embodiments, the CTL has enhanced activity and/or proliferation (e.g. enhanced cytotoxic activity) in vivo. In one embodiment, providing a CTL in vivo in step (i) comprises administering an engineered (e.g. CAR) T cell to a subject. The engineered CTL may have enhanced activity and/or proliferation (e.g. enhanced cytotoxic activity) in vivo. In a further embodiment, inhibiting or reducing the activity of one or more post-transcriptional regulators of gene expression in vivo comprises administering to the subject the machinery required for disruption of the gene encoding said post-transcriptional gene expression regulator, such as in the form of a vector (e.g. a viral vector) encoding a Cas protein together with one or more sgRNAs comprising complementary sequences to said gene encoding the post-transcriptional gene expression regulator. Administration may be systemically, e.g. enteral or parenteral, such as via intravenous infusion, or locally, such as directly into the tissue or organ to be treated/rejuvenated, e.g. by topical administration.

According to another aspect of the invention, there is provided a method of treating a disease or disorder in a subject in need thereof, said method comprising administering the CTL with enhanced activity and/or proliferative capacity defined herein to the subject, wherein said CTL recognises and is reactive to an antigen associated with the disease or disorder. In a further aspect, there is provided a method of treating a disease or disorder in a subject in need thereof, said method comprising administering the pharmaceutical composition comprising the CTL with enhanced activity and/or proliferative capacity as defined herein.

In one embodiment, the disease or disorder is cancer, and the CTL recognises and is reactive to a tumour antigen. Thus in a further embodiment, the subject is suffering from or is at risk of suffering from a tumour and/or cancer. References herein to “cancer” and “tumour” are used interchangeably herein and may refer to any solid tumour or liquid (e.g. blood) cancer. Examples of cancers will be well known to the skilled person, and cancers which are in particular amenable to treatment with engineered, such as CAR T cell, cell therapies (i.e. which may be treated using CTLs of the invention) will be readily recognised. In an alternative embodiment, the disease or disorder is an infectious disease, such as a virus infection, and the CTL recognises and is reactive to an antigen of the infectious disease vector.

Thus in certain embodiments, the CTL is an engineered CTL as described hereinbefore. In a further embodiment, the CTL is a CAR T cell. In a particular embodiment, the CTL comprises a chimeric antigen receptor (CAR) which recognises and is reactive to a tumour or cancer antigen as described hereinbefore.

It will be appreciated that references herein to a patient or subject relate equally to animals and humans and that the invention will find utility in veterinary treatment of any of the above mentioned diseases, disorders and conditions which are also present in said animals.

It will also be appreciated that references herein to “treatment” and “amelioration” include such terms as “prevention”, “reversal” and “suppression”. Furthermore, such references include administration of the CTL or composition comprising the CTL as defined herein prior to the onset of the disease or disorder, e.g. wherein the subject is at risk of the disease or disorder. Administration of the CTL or composition as defined herein may also be anticipated after the induction event of the injury, damage, disease or disorder, either before clinical presentation of said disease or disorder, or after symptoms manifest. Such references further include performing the method of enhancing the activity and/or proliferative capacity of the CTL as described herein in vivo either prior to the onset of the disease or disorder, or after the induction event of the disease or disorder.

Unless defined otherwise, all technical and scientific terms used herein have the meaning commonly understood by a person skilled in the art to which this invention belongs. As used herein, the term “about” when used herein includes up to and including 10% greater and up to and including 10% lower than the value specified, suitably up to and including 5% greater and up to and including 5% lower than the value specified, especially the value specified. The term “between” as used herein includes the values of the specified boundaries.

Throughout the specification and the claims which follow, unless the context requires otherwise, the word “comprise”, and variations thereof such as “comprises” and “comprising”, will be understood to imply the inclusion of a stated integer, step, group of integers or group of steps but not to the exclusion of any other integer, step, group of integers or group of steps.

In addition, as used herein and the appended claims, the singular forms “a”, “an” and “the” include plural referents unless the content clearly dictates otherwise. Thus, for example reference to “a CTL” includes two or more such cells, or reference to “a post-transcriptional gene expression regulator” includes two or more such regulators.

It will be understood that all embodiments described herein may be applied to all aspects of the invention and vice versa, and such combinations would be readily apparent from the description provided herein and to those skilled in the art.

Other features and advantages of the present invention will be apparent from the description provided herein. It should be understood, however, that the description and the specific examples while indicating preferred embodiments of the invention are given by way of illustration only, since various changes and modifications will become apparent to those skilled in the art. The invention will now be described using the following, non-limiting examples:

### Examples

### Example 1: Materials and Methods

Construction of PKLV2 sgRNA puro-Thyl.1 and MSCV_sRNA puro-Thyl.1

PKLV2_hU6_Bbsl-ccdB-Bbsl_iScaffold_mPGK_puro-2A-Thyl.1 (PKLV2_sgRNA_puro-Thyl.1) was generated in two rounds of cloning from Addgene plasmid 67974 (pKLV2-U6gRNA5(Bbsl)-PGKpuro2ABFP-W). Firstly, a PCR product encoding Thyl.1 and the appropriate flanking sequences was generated and ligated (Gibson assembly) into a \Xhol+\Notl linearised vector (Addgene plasmid 67974). Secondly, a PCR product encoding the ccdB toxic gene, a region validated to contain the ccdB gene's uncharacterised promoter, and the appropriate flanking sequences was generated and then ligated (Gibson assembly) into a \Bbsl linearised vector (PKLV2_hU6_bbsl-bbsl_iScaffold_mPGK_puro-2A-Thyl.1). Subsequently, the MSCV_hU6_Bbsl-ccdB-Bbsl_iScaffold_mPGK_puro-2A-Thyl.1 (MSCV_sgRNA_puro-Thyl.1) was generated in one round of cloning from MIGRI. A PCR product encoding hU6_Bbsl-ccdB-Bbsl_iScaffold_mPGK_puro-2A-Thyl.1 and the appropriate flanking sequences was generated and ligated (Gibson assembly) into a \Xhol+\Sall linearised vector (MIGRI). Note: iScaffold refers to an improved sgRNA scaffold. This was improved by removing a RNA polymerase III pause sequence (“TTTT”), and by extending the duplex region bound by Cas9.

Cloning of Individual sqRNAs into Lentivirus and Retrovirus Backbones

Method 1: two 24nt oligonucleotides were annealed and ligated (T4) into a \Bbsl linearised vector (PKLV2_sgRNA_puro-Thyl.1 or MSCV_sgRNA_puro-Thyl.1). These encoded either the complement or the reverse complement of a sgRNA seed region (19nt) with flanking sequences that formed the appropriate sticky ends.

Method 2: two 75nt oligonucleotides were annealed and ligated (Gibson assembly) into a \Mfel linearised vector (RetroQ_hU6_Mfel_iScaffold_mPGK_puro-2A-Thyl.1). These encoded either the complement or the reverse complement of a sgRNA seed region (19nt) with flanking sequences that formed blunt ends.

Method 3: individual 75nt oligonucleotides were converted to double stranded DNA by PCR, that also extended their length, and then ligated (Gibson assembly) into a \Bbsl linearised vector (PKLV2_sgRNA_puro-Thyl.1 or MSCV_sgRNA_puro-Thyl.1). These oligonucleotides were cloned to mimic both human and mouse sgRNA library construction.

Method 4: pairwise sgRNA vectors were generated by two rounds of cloning into a \EcoRI linearised vector (modified RetroQ_EcoRI-ccdB-EcoRI_mPGK_puro-2A-Thyl.1). Firstly, individual sgRNA seed regions were independently cloned using “method 1” into a donor vector, either MSCV_sgRNA_puro-Thyl.1 or a modified Psp73 vector. Each donor vector contained a human, mouse or bovine U6 promoter (hU6, mU6 and bU6) directly upstream of the cloned sgRNA seed region, and a sgRNA iScaffold directly downstream. Secondly, multiple PCR products (primers: MS1-4, template: relevant donor vector) encoding hU6_seed_iScaffold or mU6_seed_iScaffold or bU6_seed_iScaffold with the appropriate flanking sequences were generated and ligated (Gibson assembly) into a \EcoRI linearised vector (modified RetroQ).

Design of sqRNA Seed Regions

Publicly available software (“sgRNA designer”) developed and hosted online as a web portal by the Broad Institute was used to design sgRNAs for CRISPR-Cas9 knockout of targets; both individual sgRNA designs and library sgRNA designs. Settings: “Select CRISPR Enzyme” was set to “S.pyogenes NGG”, “Select Target Taxon” was set to “human” or “mouse”, “Input” was provided as NCBI Gene IDs, “Quota” was set to “10”. The sgRNA designer software provides efficiency and specificity scores for every possible sgRNA against an individual target's constitutive exons. These sgRNAs are ranked based on a combined score determined by their efficiency and specificity scores. The software recommends the most appropriate sgRNAs to choose, until the “Quota” is full. Factors that influence this include combined rank, sgRNA targeting location (inside or outside a 5%-65% region of the coding sequence), spacing from previously chosen sgRNAs, and transcriptional incompatibility due to RNA polymerase III pause sequence (“TTTT”).

Generation of Targeted Mouse RBP CRISPR-CAS9 sgRNA Libraries

The mouse sgRNA library consisted of 13500 sgRNAs. All libraries were generated with the same cloning strategy, consisting of one round of cloning into either the PKLV2_sgRNA_puro-Thyl.1 or the MSCV_sgRNA_puro-Thyl.1 backbone vectors. Single stranded oligonucleotide pools were purchased (Twist bioscience), desalted, and lyophilised at a scale of ˜0.1fmole per oligonucleotide. Oligonucleotides were invariantly 75mers that encoded a 19nt sgRNA seed region flanked by adapters. The first nucleotide of each 20nt seed region was invariantly replaced with guanine. Oligonucleotide pools were resuspended in 1× TE buffer to a final concentration of 0.25 μM. These oligonucleotide pools acted as the template, at a final concentration of 0.01UM, for a 10-cycle 25 μl PCR with an annealing temperature of 68ºC. The PCR product (150 bp) was purified and eluted in 25 μl (Zymo Research: D4005), the manufacture's protocol was followed. A purified negative control PCR with no polymerase and no template was used to determine background.

PKLV2_sgRNA_puro-Thyl.1 and MSCV_sgRNA_puro-Thyl.1 backbone vectors were digested at 37° C. overnight with 10 units of a Bbsl isoschizomer (Thermo Scientific™: ER1011, Bpil) at a scale of 10 μg in 20 μl. The digestion product (˜8,500 bp) was run on a 1% agarose (Thermo Scientific™: 16500500) gel with 1:10,000 SYBRSafe (Thermo Scientific™: S33102) and cut out quickly (≤10 seconds) under long A UV-light (UVP® M-20). The linearised plasmid was extracted (Qiagen®: 28704), the manufacture's protocol was adapted. Invariantly, gel purified DNA was further purified (Zymo Research: D4005).

Four identical 20 μl Gibson assembly reactions were simultaneously performed, each with: ˜200 ng of linearised vector (˜2 μl) (˜8500 bp) and ˜30 ng of insert (˜2 μl) (150 bp) (˜1:7.5 molar ratio). The products of these reactions were pooled together, purified and concentrated (Zymo Research: D4005), and electroporated into electrocompetent bacteria.

### Generation of Lentivirus and Retrovirus for Transduction of Human and Mouse Cell Lines and Primary Cells

HEK293FT cells were used to package pantropic lentivirus and platinum-E (PLAT-E) cells were used to package ecotropic retrovirus. Both cell lines were maintained in supplemented DMEM high glucose media (Dulbecco's modified Eagle's media) (Gibco™: 41965) (final: 10% heat inactivated (FBS) fetal bovine serum) and passaged by 1:10 dilution thrice per week from a confluent density of 10×10⁶ cells/“10 cm” plate (Nunc™: 150350), this required trypsinisation for ˜1 minute (Gibco® TrypLET: 12604-013).

### Primary Mouse T Cell Culture

Single cell suspensions of cells were prepared from the spleen and lymph nodes (inguinal, axillary, brachial, cervical) in RPMI-1640 media (Thermo Scientific™: 22409015), by passing through a 40 μM cell strainer (Falcon™: 352340). Leukocytes were enriched by the differential lysis of red blood cells with ACK lysis buffer (Thermo Scientific™: A1049201). Invariantly, tissues were harvested from mice that expressed transgenic T cell receptors designed to specifically recognise ovalbumin residues 257-264 (amino acid sequence: SIINFEKL) in the context of H2-Kb MHC class I molecules. Such mice are known as OT1 mice.

CD8 T cells were cultured either in effector cultures which induced a high rate of proliferation (˜300% clonal expansion per day), and differentiation towards cytotoxic lymphocytes (CTL).

Day0: total leukocytes were seeded at 6×10⁶ cells per well in a 12-well plate with OVA peptide and IL-2 and IL-12 cytokines in 2 ml of supplemented Iscove's Modified Dulbecco's Medium (IMDM) media with glutamax.

Day 1: total cells were transferred to a 15 ml conical tube, multiple wells were combined, cells were then centrifuged at ˜300 g for 5 minutes at room temperature, and washed twice in 5 ml of prewarmed DPBS (Dulbecco's phosphate-buffered saline), then seeded in the absence of OVA peptide and absence of IL-12 in the presence of IL-2 in supplemented IMDM media in an equivalent number of wells of a 12-well plate; if applicable total cells underwent retrovirus transduction by spinfection. On each subsequent day cells were reseeded in fresh IL-2 containing IMDM media at 1×10⁶ cells per ml. The OVA antigen specific CD8 T cells (OT1 T cells) enriched from an initial seeding proportion of ˜25% to ˜100% of viable cells by day 3.

On day 7 OT1 CD8 T cells were restimulated with 10-7M OVA (N4) peptide in the presence of 1× brefeldin A (BioLegend®: 420601) in the absence of cytokines in 100 μl of IMDM media at a density of ˜1×10⁶ cells per well of a 96-well plate.

### Tissue Culture for CRISPR-Cas9 Knock Out Screen of Primary Mouse CD8+ Cytokine Production

Single cell suspensions of leukocytes were prepared from spleen and peripheral lymph nodes separately from eight mice (R26-GFP-Cas9 CD4-Cre OT1) and seeded in effector CD8 T cell differentiation conditions. Cells from different donors were cultured independently throughout. On day 1 cells were transduced with the mouse RNA-binding protein (RBP) sgRNA library by spinfection at an MOI of 0.1 with a predetermined amount of retrovirus supernatant. On day 2, cells were harvested, and transduced cells were positively selected by MACS. On day 2, transduced CD8+ T cells were seeded and maintained in IL-2. On day 7, effector CD8+ T cells were restimulated with 10-7M OVA (N4) peptide in the presence of 1× brefeldin A for 1 hour in a T75 flask. IFN-γ positive and negative CD8+ T cells were physically separated by FACS, as were Tnf-α positive and negative cells; separation based on the concurrent expression Ifn-γ and Tnf-α was not undertaken. Enough cells (˜15×10⁶) to ensure a representation of >1000× transduced cells per sgRNA in the library in each population were enriched, washed twice in PBS, and snap frozen on dry ice in a 15 ml conical tube before storage at −80ºC for future analysis of RBP involvement in CD8 T cell cytokine production, and subsequently used as a proxy for effector CD8+ T cell differentiation and CD8+ T cell activation. In addition, on day 1 and day 7, bulk cells, unselected for transduction or cytokine production, with a >1000× representation of the sgRNA library, were processed and stored in a comparable fashion for future analysis of RBP involvement in proliferation/survival.

### Next Generation Sequencing (NGS) Library Preparation and Next Generation Sequencing

Before generating the next generation sequencing library for each sample, the optimum amount of genomic DNA (gDNA) per PCR and the minimum number of PCR cycles were determined. The amount of gDNA was titrated between 1-10 μg per PCR. The number of PCR cycles was titrated between 18-26 cycles. 2.5 μg and 22 cycles were optimum for most samples. An appropriate number of 50 μl PCRs to maintain 1000× representation of each sgRNA in the library were set up.

The PCR products were combined and concentrated (Zymo Research: D4005), the manufacture's protocol was followed. The concentrated PCR product was run on a 2% agarose gel (Thermo Scientific™: 16500500 with 1:10,000 SYBRSafe and cut out quickly (≤10 seconds) under long A UV-light. The gel slice was measured precisely (nearest 0.01g) and trimmed until 23 0.10g. Inside a 1.5 ml microcentrifuge tube (Axygen™: MCT-175-C), the gel slice was crushed, ≤3× (v/w) room temperature QG buffer was added, and subsequently incubated at room temperature for around five minutes with intermittent inversion and vortexing until the gel slice was fully dissolved. This step was critical to avoid dsDNA denaturation (data not shown). Then the manufacture's protocol was followed. The spin column was successively washed with 500 μl QG buffer, to remove excess agarose, then twice with 750 μl PE buffer, to remove excess salt. Finally, dsDNA was eluted with 30 μl EB buffer. Before each centrifugation step, the QG, PE and EB buffers were incubated with the spin column for ˜3 minutes to permit dissolution. Invariantly, gel purified DNA was further purified (Zymo Research: D4005). Individually NGS libraries were checked by nanodrop™ (Thermo Scientific™: ND-2000) and bioanalyzer (Agilent™ 2100) for purity and size, and then quantified by Kappa qPCR.

NGS libraries generated from the plasmid sgRNA libraries were ran on the MiSeq: 50 bp single read (Illumina™). NGS libraries generated from gDNA harvested during CRISPR-Cas9 knockout screens were ran on the HiSeq: 50 bp paired end read (Illumina™). Critically, the reagents in the Illumina kits were extended by 50% so the read length was 75 bp in total.

Analysis of Pooled CRISPR-Cas9 Knock Out Screens with MAGeCK

DESeq was used to determine the abundance of sgRNAs from raw fastq files. sgRNAs with an absolute count under 100 were removed. Hits in our genetic screens were identified with the MAGeCK software. The non-targeting negative control sgRNAs were used to build a mean-variance model for null distribution, which in turn was used to calculate significant sgRNA enrichment. Positive and negative enrichments were calculated, then independently used in robust rank aggregation to obtain gene-level scores. In effect this process identified the extent to which each sgRNA enriched out of the distribution of negative control sgRNAs, then ranked genes by the consistency of their sgRNAs to outperform the null hypothesis. Gene-level p-values were calculated by randomizing sgRNA to target gene allocation in permutation tests; false discovery was controlled by the Benjamini-Hochberg procedure. Log 2 fold changes (LFC) were calculated as the median LFC for all sgRNAs for a targeted gene that enriched in the same direction. Z-scores were calculated as the number of standard deviations from the mean of all negative control LFCs.

### Example 2: ZFP36 and ZFP36L1 Promote CD28 Co-Stimulation Dependence for Activation

T cell activation, proliferation and differentiation are intimately linked. A previous study suggested CD8+ T cells deficient for ZFP36 to proliferate more rapidly in response to TCR stimulation (Moore et al. (2018)). To test whether naïve isolated CD8+ T cells were more sensitive to TCR dependent activation the amount of plate-bound anti-CD3 was titrated and the proliferation of cell trace labelled Zfp36 and Zfp3611 dKO and WT cells was analysed after 72 h.

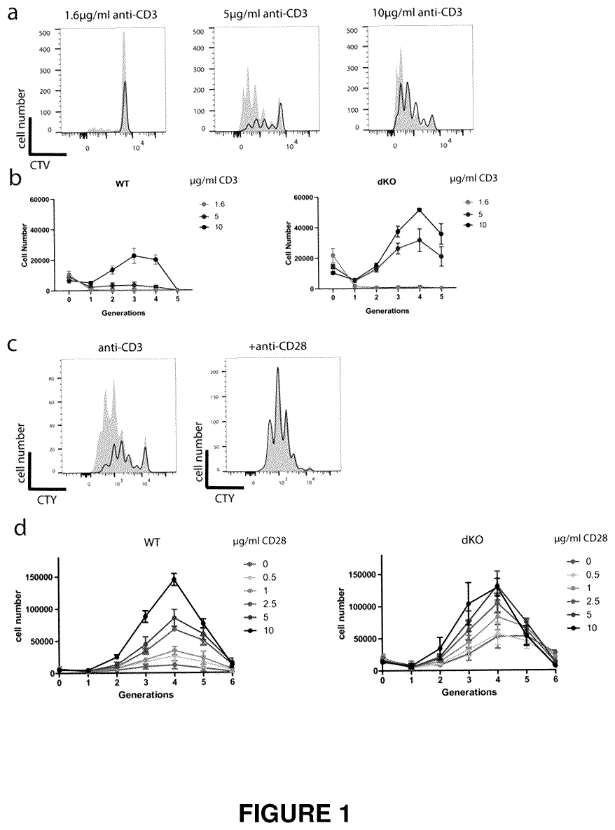

As shown in FIG. 1, dKO cells responded to lower doses of anti-CD3 with greater proliferation as compared to WT cells. Upon stimulation with higher doses of anti-CD3 the dKO cells had still a proliferative advantage as compared to the WT cells (FIGS. 1A-1C). Co-stimulation by cytokines and cell surface ligand-receptor interactions (e.g. CD28) is critical for naive CD8+ T cell activation and subsequent differentiation. Therefore, the co-stimulation dependence of dKO CD8+ T cells was investigated by the titration of anti-CD28 antibodies. Following stimulation with 5 μg/ml plate bound anti-CD3, cell trace labelled dKO CD8+ T cells divide more than WT cells (FIG. 1D). The addition of anti-CD28 (clone 37.51) increased the numbers of both, WT and dKO cells present after 72 h culture, compared to cultures with anti-CD3 alone (FIG. 1D). WT cells show a greater sensitivity to lower amounts of CD28 costimulation compared to the dKO CD8+ T cells. The latter are only mildly responsive to the inclusion of anti-CD28 as measured by the absolute cell numbers in the cultures after 72 h with LogEC₅₀ of 2.2 μg/ml for WT and 1.2 μg/ml dKO cells (data not shown). dKO cells accumulate more cells per generation, when stimulated with CD3 alone as compared to their WT counterparts (data not shown), which results in a reduced sensitivity to additional CD28 co-stimulation in dKO cells. This contrasts with WT cells which are critically dependent on signals from CD28, which opposes the inhibition of activation by ZFP36 and ZFP36L1, to enable a robust proliferative response.

### Example 3: Cytotoxic Cell Killing is Increased in ZFP36 and ZFP36L1 dKO Cells

To test the effector function of CTLs with deletions of the RNA-binding proteins ZFP36 and ZFP36L1, dKO CD8+ CTLs were incubated with target cells loaded with either high (N4) or low (V4) affinity peptide. Transgenic OT-I CTLs with TCRs which recognise OVA peptide were used.

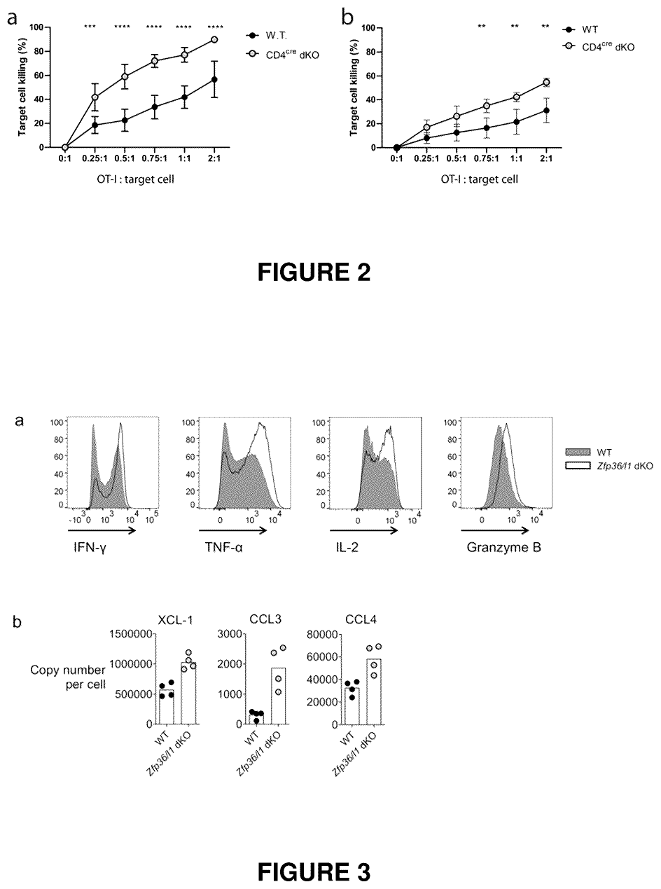

When differentiated to CTLs, CD8+OT-I T cells lacking ZFP36 and ZFP36L1 were better killers of cells presenting high affinity peptide MHC (FIG. 2A) or with a peptide-MHC of 100-fold lower potency (FIG. 2B). As shown in FIG. 3, when differentiated in vitro to a memory-like phenotype using IL-7, CD8+ cells lacking ZFP36 and ZFP36L1 produced increased amounts of TNFα, IFNγ and IL-2 (FIG. 3A) and also chemokines CCL3, CCL4 and XCL1 (FIG. 3B) after stimulation with antigen.

### Example 4: Genetic Screen of RNA-Binding Proteins that Regulate CD8+ T Cell Activation

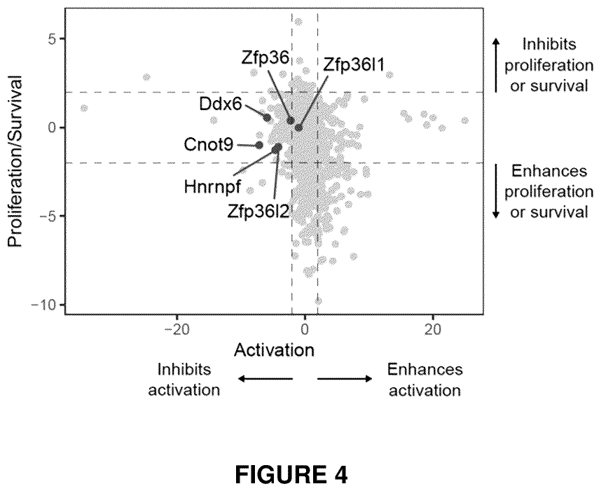

RBPs that promote or inhibit CD8+ T cell activation were identified by comparing genetic screens performed for TNFα and IFNγ production of CD8+ T cells after stimulation with antigen. This strategy involved identifying RBPs that were enriched or depleted to a comparable extent in both screens. Overall, 44 RBPs significantly enriched for a role in limiting CD8+ T cell activation (effector cytokine production) independently from a role in activating proliferation or survival (Table 1). Amongst the most enriched were the Ddx6 gene encoding an RNA helicase; Hnrnpf encoding heterogeneous nuclear ribonucleoprotein F; and Cnot-4, -6l and -9 components of the mega-Dalton CCR4-NOT complex (FIG. 4). All are associated with the function of the ZFP36 family, supported by published crystallography for CCR4-NOT.

Taken together, these results indicate the role of RNA-binding proteins (RBPs) in limiting the activation and effector functions of CTLs, in particular CD8+ T cells, and the ability to enhance the activity (e.g. effector cytokine production and cytotoxic killing ability) of CTLs by deleting particular RBP genes.

### Example 5: Cytotoxic Cell Function (Killing) is Enhanced Following Knock Out of Ddx6 and Cnot9

### Primary Mouse T Cell Culture

Naïve OT-I cells were isolated from Spleen and peripheral lymph nodes of female B6SJL mice (8-12 weeks old), using EasySep Mouse Naïve CD8+ T Cell Isolation Kit (StemCell:19858), according to the manufacturer's protocol.

CRISPR/Cas9 gene editing of one million naïve OT-I cells was conducted according to Nussing et al., (2022) J. Immunol. (2020) ji1901396. In brief, naïve cells were suspended in room temperature OptiMEM reduced serum media (ThemoFisher: 31985070) at a density of ten million per ml, and three μM of CrRNA:tracrRNA+ one μM Cas9 protein (IDT: 1081059) were electroporated into cells using NEPA21 Super Electroporator (NepaGene); see Table 2 for poring pulse and transfer pulse conditions. Cells were electroporated with Ddx6, Cnot9-targeting or non-targeting control CrRNA:tracrRNA; see Table 3 for single-guide RNA seed sequence. Following electroporation, cells were washed with 15 ml Iscove's Modified Dulbecco's Medium (IMDM) (ThemoFisher: 31980030, supplemented with 10% heat inactivated foetal calf serum (FCS), 100 U/ml Penicillin/Streptomycin (ThermoFischer: 15140130), and 50 μM 2-mercaptoethanol (ThermoFischer: 31350-010), cells were centrifuged at ˜300 g for 5 minutes at room temperature.

Following washing, cells were re-suspended at one million per ml of supplemented IMDM, and were maintained in culture (37° C., 5% CO₂) for 48 hours in the presence of 10 ng/ml of recombinant murine Interleukin-7 (rmIL-7) (Peprotech:217-17), in a 48 well plate (Nunc: 150687).

Cytotoxic T cell cultures were conducted by activating cells 48-hours post electroporation using WT B6SJL splenocytes pulsed with 100 nM SIINFEKL peptide (Genscript RP10611), in the presence of 20 μg/ml rmIL-2 and 2 μg/ml rmIL-12 (PeproTech: 212-12 and 210-12), diluted in supplemented IMDM. Electroporated cells were seeded at a 1:4 ratio with pulsed splenocytes and co-cultured for 48 hours (37° C., 5% CO₂) in a 24-well plate (Nunc; 142475), following which cells were harvested and washed twice with warm media (˜300 g for 5 minutes at room temperature), before being returned to culture at a density of 0.5 million per ml with 20 ng/ml IL-2, in a 6-well plate (Nunc; 140675). Cells were expanded for 5 days by splitting every 24 hours at a 1:2 ratio and providing the cells with fresh IL-2. Cells expanded approximately 100-fold using this culture method. Cell numbers and diameter were calculated using CASY Model TT cell counter (Scharfe systems/Roche), 10 μl of cell suspension was added to 10 ml of CASY ton (1 in 1000 dilution). Cells between 7.5-30-13 μm in diameter were measured.

### Target Cell Killing Assay

EL4 cells (ATCC: TIB-39) were cultured in RPMI supplemented with 1× GlutaMAX™ (Gibco®: 35050061), 100 U/ml Pencillin, 100 μg/ml Streptomycin (Thermo Scientific™: 15140) and 10% FCS and passaged by 1:20 dilution twice per week from a confluent density of 10×10⁶ cells/T75 flask (Nunc: 156472). EL4 cells were labelled with CellTrace™ Violet (ThermoFisher™; C34557) or CellTrace™ Yellow (ThermoFisher™; C34567) dye following the manufacturer's protocol. Target EL4 cells were labelled using 10 μM CellTrace, and non-target cells were labelled using 0.5 μM CellTrace. Target EL4 cells were pulsed with 100 nM SIINFEKL by incubating for 30 min at 37° C. at a density of one million cells per ml of RPMI in a conical tube. Non-target EL4 cells were incubated with vehicle control (1:10,000 DMSO, Sigma:D2650). Following incubation EL4 cells were underlaid with 3.5 ml room temperature FBS. Cells were then washed 2× with RPMI; cells were centrifuged at ˜300 g for 5 minutes at room temperature.

CTLs: target: non-target cells were seeded at ratios ranging from 2:1:1 to 0.125:1:1 in round-bottomed 96 well plates (Nunc: 262162) in 200 μl supplemented IMDM, and incubated for 3 hours (37° C., 5% CO₂). Cells number and diameter were calculated using CASY Model TT cell counter. For analysis of cytokine expression by intracellular flow cytometry, 1× Brefeldin A (eBioscience: 00-4506-51) secretion inhibitor was added to cells for the final hour of culture. Target cell killing experiments were stopped by maintaining cells on ice until cells were fixed for flow cytometry analysis.

Target cell killing (%) was calculated using the following equation: 100-{(percentage of targets/percentage non-targets)/(time 0 percentage of targets/time 0 percentage of non-targets) ×100}. Cell numbers were calculated during flow cytometry analysis by adding 1.25 μl of accurate count 70 μm fluorescent beads (Saxon Europe: ACBP-50-10) per 100 μl of MACS buffer (equivalent to 1,270 beads).

### Flow Cytometry

Cell suspensions were prepared in MACS buffer (DPBS+2% FBS (v/v) 2 mM EDTA), and stained with fixable viability dye eFluor780 (eBioscience: 65-0865-14) diluted 1:5000, while simultaneously blocking with anti-Fc receptor (CD16/32) antibody (Clone: 2.4G2, BioXcell: BE0307) diluted 1:5000, for 20 minutes on ice. For intracellular cytokine and effector molecule staining, cells were fixed using BD Cytofix/Cytoperm (Beckinson Dickinson: 51-2090kz) on ice for 30 min and stained for 60 min at room temperature with antibody solutions diluted in 1× BD PermWash buffer (Beckinson Dickinson: 51-2091kz). Flow cytometry analysis on stained cells in FACS buffer was performed using BD LSRFortessa (Beckinson Dickinson). FlowJo V10.6.0 was used for data analysis (Beckinson Dickinson). A comprehensive list of antibodies used for flow cytometry analysis are listed in Table 4.

### Immunoblotting

Two million day 7 CTLs were harvested and centrifuged at 330g for 5 minutes at 4° C. and washed twice with 5 ml of ice-cold phosphate buffered saline (PBS) before re-suspending in 20 μl PBS and transfer to 1.5 ml Eppendorf. Lysates were prepared by adding equal volume of 2×RIPA buffer supplemented with 1:50 protease inhibitor cocktail (Sigma: P8340). Cells were then vortexed and incubated on ice for 8 minutes, before centrifuging at 20,000g for 12 minutes at 4ºC. Protein lysate concentration was determined using Pierce BCA protein assay kit according to the manufacturer's protocol (Invitrogen: 23225). Laemmli buffer supplemented with 5% 2-mercaptoethanol was then added at a 1:4 v/v ratio to lysates, and lysates were then denatured for 5 min at 97° C.

The equivalent of 40 μg of total protein was loaded per lane of 10% SDS-PAGE gels prepared using Bio-Rad mini-protean assembly apparatus (Bio-Rad:165-8000) and proteins were resolved by running at 120 V for ˜1.5 hours. Proteins were then transferred to nitrocellulose membranes using semi-dry electrophoretic transfer apparatus (Biometre). Proteins were transferred at 3.5 mA/cm² (constant current) for 45 minutes. Nitrocellulose membranes were subsequently incubated in intercept PBS blocking buffer (Li-Cor: 927-90001) o/n at 4° C. Following block, membranes were incubated with primary antibodies diluted in intercept buffer for o/n at 4° C. (see Table 5 for details). Nitrocellulose membranes were subsequently washed three times with TBS-T (0.05% Tween-20), and three times with TBS, before being incubated with secondary antibodies diluted in intercept buffer for 2 hours at room temperature (see Table 6 for details). Nitrocellulose membranes were subsequently washed three times with TBS-T (0.05% Tween-20), and three times with TBS. Membranes were scanned using Image Li-Cor odyssey DLx scanner and analysed using ImageStudio Lite version 5.2 (Li-Cor).

TABLE 2
Parameters for NEPA21 Super Electroporator
Set Parameters

| Poring Pulse | | | | | | Transfer Pulse | | | | | |
|---|---|---|---|---|---|---|---|---|---|---|---|
| | Length | Interval | | D. Rate | | | Length | Interval | | D. Rate | |
| V | (ms) | (ms) | No. | (%) | Polarity | V | (ms) | (ms) | No. | (%) | Polarity |
| 275 | 2 | 50 | 2 | 10 | + | 20 | 50 | 50 | 5 | 40 | +/− |

TABLE 3
IDT single-guide RNA seed region sequences

| sgRNA target | sgRNA seed region 5′-3′ |
|---|---|
| Ddx6 | CATGTGGTGATCGCTACCCC |
| Cnot9 | CCAAATGAATGCCATAGCAT |
| Non-target | GCACTACCAGAGCTAACTCA |

TABLE 4
Fluorophore-conjugated antibodies used for flow cytometry

| Epitope | Conjugation | Clone | Dilution | Isotype | Source |
|---|---|---|---|---|---|
| CD8a | BUV395 | 53-6.7 | 1:400 | Rat | BD |
| | | | | IgG2a | Pharmigen: |
| | | | | | 563786 |
| Granzyme | AF647 | GB11 | 1:400 | Mouse | BioLegend: |
| B | | | | IgG1 | 515406 |
| IFN-γ | PE-Cy7 | XMG1.2 | 1:800 | Rat | BioLegend: |
| | | | | IgG1, k | 505826 |
| IL-2 | PE | JES6-5H4 | 1:200 | Rat | BioLegend: |
| | | | | IgG2b k | 503808 |
| Perforin | PE | S16009B | 1:800 | Rat | BioLegend: |
| | | | | IgG2a | 154406 |
| TNF-α | BV650 | MP6-XT22 | 1:400 | Rat | BioLegend: |
| | | | | IgG1, k | 506333 |

TABLE 5
Primary Antibodies used for immunoblotting

| | | | | Dilution |
|---|---|---|---|---|
| Epitope | Clone/Name | Isotype | Source | WB |
| DDX6 | | Rb IgG | CST: | 1:1,000 |
| | | | 9407 | |
| CNOT9 | | Rb IgG | Proteintech: | 1:5,000 |
| | | | 22503-1-AP | |
| β-actin | AC-15 | Ms IgG1 | Sigma: | 1:10,000 |
| | | | A1978 | |

TABLE 6
Secondary Antibodies used for immunoblotting

| Epitope | Isotype | Conjugate | Dilution | Source |
|---|---|---|---|---|
| Rabbit Ig H + L | Goat IgG | IRdye | 1:10,000 | Li-Cor: |
| | | 680 RD | | 926-68071 |
| Rabbit IgG | Donkey IgG | IRDye | 1:10,000 | Li-Cor: |
| | | 800 WC | | 925-32213 |
| Mouse Ig H + L | Donkey IgG | IRDye | 1:10,000 | Li-Cor: |
| | | 680RD | | 926-68072 |
| Mouse Ig H + L | Goat IgG | IRDye | 1:10,000 | Li-Cor: |
| | | 800 WC | | 925-32210 |

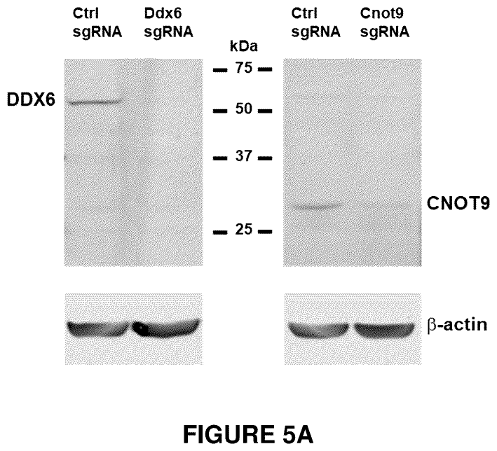

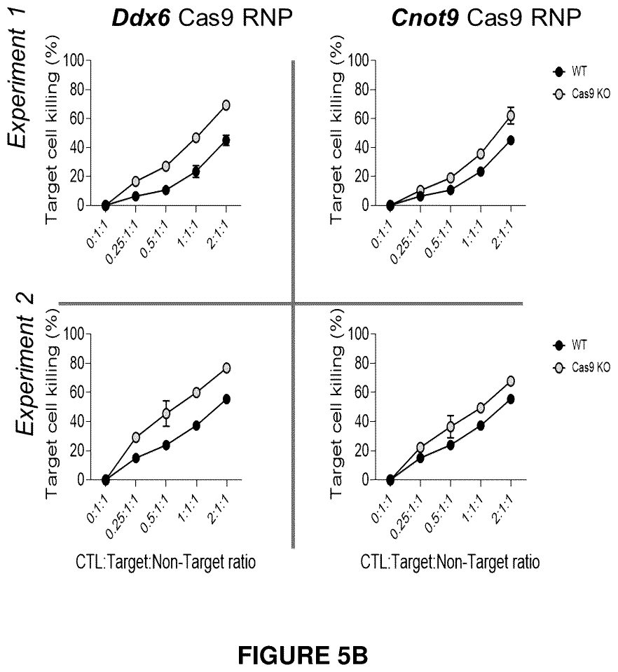

By immunoblotting using Rabbit polyclonal anti-DDX6 (CST: 9407) or anti-CNOT9 (Proteintech 22503-1-AP) antibodies, we demonstrated that genetic deletion of Ddx6 and Cnot9 in naïve CD8 T cells by electroporation of Cas9 and sgRNA leads to a near-total loss of DDX6 and CNOT9 protein in differentiated cytotoxic T lymphocytes (CTLs) (FIG. 5A). These results indicate that naïve CD8 T cells which feature Cas9-mediated genetic deletion of either RBP are under no survival disadvantage when expanded using out CTL culture method.

Using our flow cytometry-based cytotoxicity assay, we found that CTLs which lack DDX6 or CNOT9 are significantly improved killers of target cells presenting N4-OVA-peptide (FIG. 5B). When seeded at a 2:1:1 ratio, Ddx6 KO CTLs could kill 67% (+6), and Cnot9 KO CTLs could kill 56% (+7) of N4-presenting target cells, over two independent experiments. In comparison CTLs treated with non-targeting control sgRNAs could kill 42% (+4) of target cells. These results indicate that the RBPs DDX6 and CNOT9 act to limit CD8 T cell cytotoxicity at a cell-intrinsic level.

### Example 6: DDX6 Limits Cytotoxic Protein Expression by Cytotoxic T Lymphocytes (CTL) and DDX6 KO OT-I Cells May Provide Increased Protection from IAV Infection DDX6 Limits Cytotoxic Protein Expression by CTL

To expand on the cytotoxicity assay results of Example 5, proteomes of CTL derived from naïve OT-I cells electroporated with Cas9 and control (Ctrl)-or Ddx6-targeting sgRNA were analysed using single-shot mass-spectrometry, and protein copy numbers per cell were estimated using “histone ruler” methodology. CTL were taken at day 7 and were either resting or stimulated with 0.01 nM N4 peptide for one or three hours.

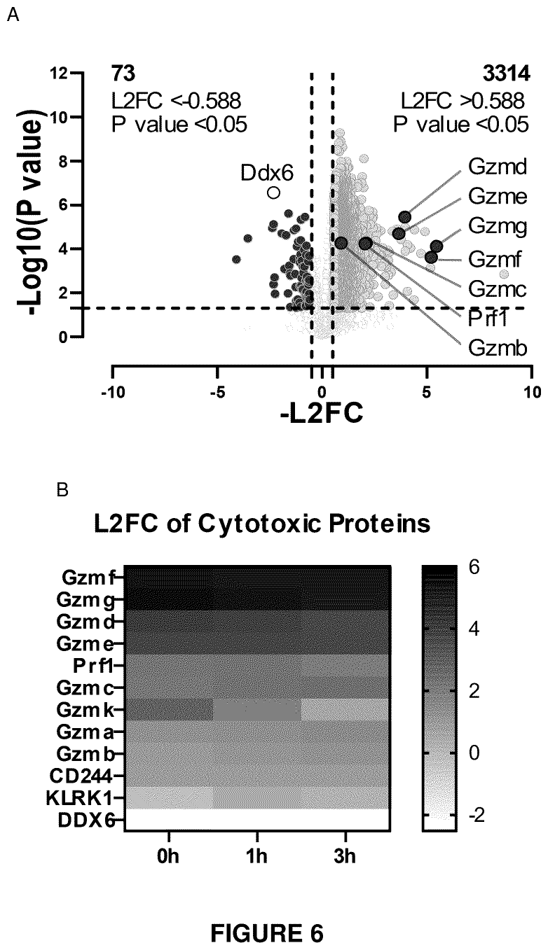

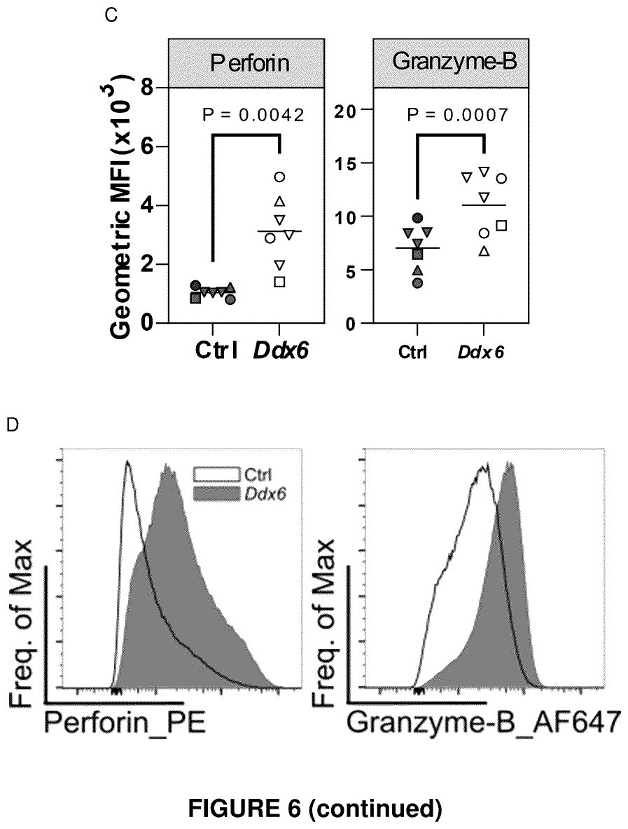

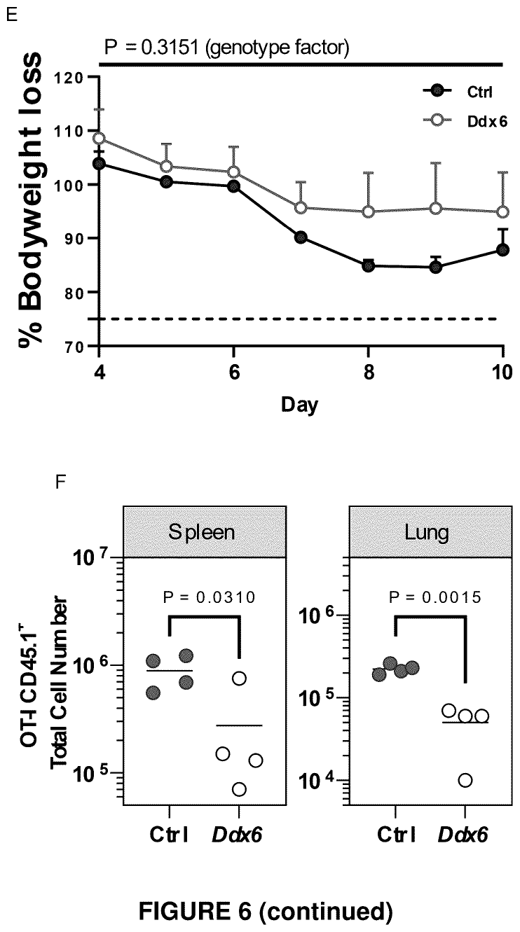

FIG. 6A demonstrates the change in protein abundance in DDX6 KO CTL compared to Ctrl CTL. DDX6 abundance is downregulated as expected, while a number of proteins involved in cytotoxicity are increased in abundance. This is confirmed in FIG. 6B where the protein copy number of cytotoxic proteins is seen to increase in DDX6 KO CTL compared to Ctrl CTL following stimulation, and in FIGS. 6C and 6D which show the increased amounts of the Pore-forming protein Perforin and cytotoxic serine protease Granzyme-B in DDX6 KO CTL compared to Ctrl CTL.

These data indicate that DDX6 limits the expression/abundance of cytotoxic proteins in CTL and the enhanced killing function demonstrated in Example 5 is likely due to an increased abundance of these proteins in DDX6 KO CTL compared to Ctrl CTL, in particular upon stimulation.

DDX6 KO OT-I Cells May Provide Increased Protection from IAV Infection, while Limiting the Number of Cells Recovered from Draining and Infected Tissues

To determine the effects of enhanced cytotoxic activity of CTL in vivo, two hundred naïve OT-I cells electroporated with Cas9 and control (Ctrl)- or Ddx6-targeting sgRNA were adoptively transferred into congenically marked (CD45.2+) recipient B6.SJL mice. Recipients were challenged 24 hours post-transfer of cells with 1000 PFU of H1N1 Influenza A Virus (IAV) expressing SIINFEKL peptide (WSN-OVA) intranasally.

FIG. 6E shows that mice transferred with Ddx6 KO CTL displayed less bodyweight loss post-infection than those transferred with Ctrl CTL. This is correlated with fewer Ddx6 KO CTL recovered from the spleen and lungs of these mice compared to Ctrl (FIG. 6F), indicating a reduced response of these cells upon infection and resulting protection from detrimental effects of infection.

1. A method of enhancing cytotoxic T lymphocyte (CTL) activation and/or proliferation, said method comprising inhibiting or reducing the activity of one or more post-transcriptional regulators of gene expression.

2. The method of claim 1, wherein the cytotoxic T lymphocyte (CTL) is a CD8+ T cell, or is a CD4+ T cell.

3. (canceled)

4. The method of claim 1, wherein the method enhances CTL activation independently of enhancing proliferation.

5. The method of claim 4, wherein the method enhances CTL activation without enhancing proliferation.

6. The method of claim 1, wherein the effector functions of the CTL are enhanced, and/or the killing functions of the CTL are enhanced.

7. (canceled)

8. The method of claim 1, wherein the one or more post-transcriptional regulators of gene expression are one or more RNA-binding proteins.

9. The method of claim 8, wherein the one or more RNA-binding proteins regulate the stability and/or translation efficiency of an mRNA transcript.

10. The method of claim 9, wherein the one or more RNA-binding proteins reduce or inhibit the stability and/or translation efficiency of the mRNA transcript.

11. The method of claim 1, wherein the one or more post-transcriptional regulators of gene expression is selected from any one or more of those listed in Table 1, and/or is one or more components of the CCR4-NOT complex.

12. (canceled)

13. The method of claim 1, wherein the one or more components of the CCR4-NOT complex are selected from one or more of: DDX6; hnRNPF; CNOT4; CNOT6L; and/or CNOT9.

14. The method of claim 1, wherein the CTL is an engineered T cell, such as a chimeric antigen receptor (CAR) T cell.

15. The method of claim 14, wherein the engineered T cell recognises and is reactive to a tumour antigen.

16. The method of claim 1, wherein inhibiting or reducing the activity of one or more post-transcriptional regulators of gene expression comprises disrupting the gene encoding said one or more post-transcriptional regulators of gene expression.

17. The method of claim 16, wherein disruption of the gene encoding the one or more post-transcriptional regulators of gene expression comprises deletion, knock-out or introduction of an indel, and/or
wherein the gene encoding the one or more post-transcriptional regulators of gene expression is endogenous to the CTL.

18. (canceled)

19. The method of claim 1, wherein the method is performed in vitro.

20. A cytotoxic T lymphocyte (CTL) with enhanced activity and/or proliferative capacity obtainable by the method of of claim 1.

21. A method of treating a disease or disorder in a subject in need thereof, said method comprising administering the CTL with enhanced activity and/or proliferative capacity of claim 20 to the subject, wherein said CTL recognises and is reactive to an antigen associated with the disease or disorder.

22. The method of claim 21, wherein the disease or disorder is cancer, and wherein the CTL recognises and is reactive to a tumour antigen, or wherein the disease or disorder is an infectious disease, and wherein the CTL recognises and is reactive to an antigen of the infectious disease vector.

23. (canceled)

24. The method of claim 6, wherein the enhanced effector function is the release of granzyme B, perforin, TNFα, IFNγ and/or IL-2.

25. The method of claim 22, wherein the infectious disease is a virus infection.
